(19) United States
(12) Patent Application Publication
(10) Pub. No.: US 2024/0189476 A1
(43) Pub. Date: Jun. 13, 2024

(54) PHOTO-CROSSLINKABLE PLANT-BASED MATERIALS, METHODS OF MANUFACTURE THEREOF AND ARTICLES COMPRISING THE SAME

(71) Applicant: University of Connecticut, Farmington, CT (US)

(72) Inventors: Ali Tamayol, Providence, RI (US); Mohamadmahdi Samandari, Newington, CT (US); Farnoosh Saeedinejad, Hartford, CT (US); Jacob P. Quint, New Britain, CT (US); Elmira Arab Tehrany-Kahn, Nancy (FR); Cyril Kahn, Nancy (FR)

(21) Appl. No.: 18/535,288

(22) Filed: Dec. 11, 2023

Related U.S. Application Data
(60) Provisional application No. 63/431,523, filed on Dec. 9, 2022.

(51) Int. Cl. A61L 27/22; A61L 27/56; A61L 27/58; B33Y 70/00

(52) U.S. Cl. CPC A61L 27/227; A61L 27/56; A61L 27/58; B33Y 70/00

(57) ABSTRACT

A biomaterial composition includes an uncrosslinked plant-based biomaterial functionalized with reactive groups that are operative to undergo a crosslinking reaction via free radical polymerization. A method for producing a biomaterial composition includes functionalizing an uncrosslinked plant-based biomaterial with reactive groups that are operative to undergo a crosslinking reaction via free radical polymerization to form a functionalized uncrosslinked plant-based biomaterial. Additional embodiments are provided.

### CROSS-REFERENCE TO RELATED APPLICATIONS

This disclosure claims priority to U.S. Provisional Application No. 63/431,523 filed on Dec. 9, 2022, the entire contents of which are hereby incorporated by reference.

### BACKGROUND

This disclosure relates to photo-crosslinkable plant-based materials, methods of manufacture thereof and to articles comprising the same. In particular, this disclosure relates to the use of photo-crosslinkable plant-based materials for the manufacture of scaffolds that may be used for tissue engineering, food materials, and the like.

Regenerative medicine is a broad field that includes tissue engineering but also incorporates research on self-healing—where the body uses its own systems, sometimes with help foreign biological material to recreate cells and rebuild tissues and organs. Foreign biological materials (hereinafter “biological substitutes” in regenerative medicine refer to materials or constructs that are used to replace or repair damaged or lost tissues and organs within the body. These biological substitutes aim to harness the body's natural regenerative capacities or provide support for regenerative processes.

As scientists develop biological substitutes for regenerative medicine, tissue engineering has continued to evolve. Most human and animal tissues are anchorage-dependent, residing in a solid matrix referred to as an “extracellular matrix (ECM)”. The ECM is a complex three-dimensional network of proteins, carbohydrates, and other molecules that provide structural and biochemical support to the cells within tissues and organs. Natural biomaterials that have characteristics that are desirable for an ECM are usually derived from animals, which makes them unfavorable due to the ethical, environmental, and human health challenges associated with raising and slaughtering livestock. It is therefore desirable to have simple low-cost biocompatible and scalable strategies for biomaterials that are not derived from animal-based materials for scaffolding and serum-based culture media in tissue engineering.

### SUMMARY

Disclosed herein is a biomaterial composition comprising an uncrosslinked plant-based biomaterial functionalized with reactive groups that are operative to undergo a crosslinking reaction via free radical polymerization.

Disclosed herein too is a method for producing a biomaterial composition, the method comprising functionalizing an uncrosslinked plant-based biomaterial with reactive groups that are operative to undergo a crosslinking reaction via free radical polymerization to form a functionalized uncrosslinked plant-based biomaterial.

### BRIEF DESCRIPTION OF THE DRAWINGS

FIG. 1 is a flow chart that depicts an exemplary embodiment of a method for fabricating biomaterials as disclosed herein;

FIG. 2 is a depiction of an exemplary production process for the manufacture of photo-crosslinkable plant-derived biomaterials;

FIG. 3 is a bar graph that depicts protein content in the biomaterial after functionalization;

FIG. 4 depicts crosslinking of the biopolymer of FIG. 1 using electromagnetic radiation;

FIGS. 5 and 6 are graphical depictions that demonstrate the capability of a carbonate/bicarbonate buffer at different molarities to adjust the pH to be in a desirable range during the extraction;

FIG. 7A is a bar graph that depicts that a higher level of methacrylation (a higher level of functionalization) results in a lower concentration of biomaterial that may be used for crosslinking;

FIG. 7B is a bar graph that depicts that a higher level of functionalization (methacrylated protein) in a given concentration of biomaterial results in increasing stiffness for the crosslinked composition;

FIG. 7C is a scanning electron micrograph that demonstrates that the scaffold structure is highly porous and can support cellular activity and enable rapid mass transport inside the scaffold;

FIG. 8A is a micrograph that depicts live/dead staining. It demonstrates viability and morphology of myoblasts, one day after seeding on the photo-crosslinked chickpea-derived scaffold;

FIG. 8B is a bar graph that depicts quantification of the stained samples thereby demonstrating a greater than 94% viability one day after post-seeding;

FIG. 9 depicts the morphology of a stable crosslinked foam produced from a syringe and printed with simultaneous photocrosslinking using ultraviolet radiation. This demonstrates the biofabrication capability of the foam bioink;

FIG. 10A is a bar graph that depicts cell proliferation in the biomaterials (3D scaffolds) having different protein concentrations (7.5, 10 and 12.5% w/v) in the composition as a function of time (1 day, 2 days, 3 days and 6 days); and

FIG. 10B contains photomicrographs that depict live/dead staining of the cells encapsulated within the 3D scaffold at different concentrations of the biomaterial.

### DETAILED DESCRIPTION

Disclosed herein is a composition for manufacturing a construct (e.g., a scaffold) that can be used to develop engineered tissue. The construct may be for use as a prosthetic in the body of a living being or for food biomanufacturing. In one embodiment, the composition includes an uncrosslinked plant-based biomaterial functionalized with reactive groups that can undergo a reaction via a free-radical polymerization when activated by electromagnetic radiation, heat transfer, or a combination of electromagnetic radiation and heat transfer. The reaction results in crosslinking the hitherto uncrosslinked plant-based biomaterial. In an embodiment, the electromagnetic radiation is light in the visible wavelength regime (i.e., visible light). The composition can optionally include a photoinitiator to facilitate crosslinking. Disclosed herein too is a reaction product of the aforementioned composition. The reaction product includes a crosslinked plant-based biomaterial and may include a crosslinked interpenetrating network that includes two or more different proteins.

Generally, the term “autologous cells” refers to cells that are derived from and used in the same individual. These cells are obtained from the person's own body, and they are typically used for various medical procedures or therapies. The use of autologous cells has several advantages, including a lower risk of immune rejection since the cells are recognized as “self” by the immune system.

The term “additive manufacturing (AM),” also known as 3D printing, is a manufacturing process that builds objects layer by layer, based on a digital model. Unlike traditional subtractive manufacturing methods that involve cutting, machining, or molding materials to create a final product, additive manufacturing adds material layer by layer to construct the desired object.

“Visible light” refers to light in the visible portion of the electromagnetic spectrum.

In an embodiment, the reaction product of the composition may include a crosslinked, interconnected porous, construct. The porous construct can be used as a scaffold to facilitate the growth and proliferation of a variety of cells. In some embodiments, the cells can be therapeutic cells, including stem cells, which may be esophageal epithelial cells, adipose derived mesenchymal stromal cells, muscle progenitor cells, neural progenitor cells, induced pluripotent stem cells, bone marrow stem cells, or a combination thereof. In an embodiment, the aforementioned therapeutic cells may be autologous cells. In some other embodiments, the cells can be food-related species, including muscle and muscle progenitor cells, adipose or adipose progenitor cells, fibroblasts, or similar cells. Finally, the biomaterial can be formed into acellular constructs for direct food production.

Plant-based biomaterial constructs offer several advantages in various biomedical applications. Constructs derived from plant sources can serve as supportive structures for cell growth, tissue engineering, and regenerative medicine. The advantages include biocompatibility. Plant-based biomaterials are often biocompatible, meaning they are well-tolerated by living tissues. This is important for their use in medical applications, as the scaffolds can interact favorably with the surrounding biological environment without causing adverse reactions. Plant-based materials tend to have lower immunogenicity compared to some synthetic materials. This means that they are less likely to trigger an immune response in the body, reducing the risk of rejection when used in tissue engineering or implantation.

Plant-based biomaterials are derived from renewable resources, making them more sustainable than some synthetic alternatives. Some plant-based materials contain bioactive compounds that can promote cell growth and tissue regeneration. These compounds may have inherent properties that benefit the intended biomedical application, such as anti-inflammatory or antimicrobial effects. Plant-based biomaterials come from a wide variety of plant sources, offering a diverse range of structural properties. This diversity allows researchers to choose materials with specific mechanical, chemical, and biological characteristics tailored to the requirements of a particular application. Many plant-based materials are biodegradable, meaning they can break down naturally over time. This property is advantageous for temporary constructs used in tissue engineering as the constructs can gradually degrade as the new tissue grows and replaces it.

Plant-based biomaterials are often amenable to various processing techniques, such as electrospinning, freeze-drying, and 3D printing. This flexibility in processing methods allows for the creation of constructs with specific structures and properties suitable for different applications. Plant-based biomaterials can be cost-effective compared to some synthetic alternatives. The availability of raw materials and the simplicity of processing methods contribute to the economic feasibility of using plant-derived constructs in certain applications.

As noted above, the extracellular matrix (ECM) is a complex three-dimensional network of proteins, carbohydrates, and other molecules that provide structural and biochemical support to the cells within tissues and organs. ECM is a dynamic and essential component of the cellular microenvironment, playing a crucial role in various biological processes, including cell adhesion, signaling, and tissue development. The ECM gives the tissue structural and mechanical properties such as rigidity and elasticity that are associated with tissue functions. For example, well-organized thick bundles of collagen in tendon tissues are highly resistant to stretching and are responsible for the high tensile strength of tendons. On the other hand, randomly distributed collagen fibrils and elastin fibers are responsible for the toughness and elasticity of the skin.

It is desirable for a construct that is used for cell culturing to mimic a native ECM as much as possible and offer the following features (i) biocompatibility with the precursors that are used to manufacture a crosslinked construct, the crosslinked construct and any by-products resulting from the degradation of the crosslinked construct, (ii) cell binding and degradation sites, (iii) processibility from a variety of different bio-fabrication processes, (iv) adjustable mechanical properties, (v) controllable porous microstructure, and (vi) affordability and accessibility. Various natural and synthetic biomaterials have been developed to date to form scaffolding platforms for cell culture in different tissue engineering applications. However, these biomaterials fail to address all of the desirable features.

Specifically, most of the synthetic biomaterials lack proper cell permissibility into a scaffold due to their lack of innate adhesion moieties, degradation sites, or microarchitecture. Cell permissibility is the materials ability to allow cells to migrate into a biomaterial scaffold or to remodel it as the tissue develops and regenerates. The lack of proper cell permissibility prevents cellular spreading and migration in most synthetic constructs. Synthetic biomaterials can be chemically modified to be degradable and have adhesion moieties, but is an expensive and time-intensive process.

Conversely, natural biomaterials have high cell permissibility and enable cellular migration and remodeling but are usually derived from animals, which makes them unfavorable due to the ethical, environmental, and human health challenges associated with raising and slaughtering livestock, immunological concerns of xeno-based materials in regenerative medicine, and the goals of food biomanufacturing to eliminate reliance on livestock. Additionally, human-derived biomaterials cannot be applied for food biomanufacturing and are not easily accessible, are unaffordable, and risk immunological reactions for healthcare applications.

To be economically viable and commercially realizable, food biomanufacturing must be scalable, low-cost, and nutritious. Current methods for 3D cultured meat production originate from medical tissue engineering research and heavily rely on animal-derived biomaterials and culture media. While most animal-derived biomaterials offer high biocompatibility, cell adhesion and protease-based biodegradability, they contradict one of the main purposes of cell agriculture, minimizing the implementation and use of animal-derived materials. These biomaterials and serum-based reagents are also expensive and non-sustainable thus negatively impacting the scaling of cultured meat production.

In an embodiment, the composition can be blended with an aqueous solution and manufactured into a bioink that can be used in various bio-fabrication processes developed for tissue engineering and drug delivery applications. The composition is robust and can be subjected to a variety of different manufacturing processes such as for example, micromolding or bioprinting, and retains its cellular viability during and after these biofabrication processes. The biofabricating may include additive manufacturing of the porous construct by extrusion-based additive manufacturing where a model includes instructions for depositing material layer-by-layer to create a three-dimensional object. The biomaterial may be used for stereolithography-based printing, in which light facilitates crosslinking specific locations of the uncrosslinked biomaterial based on a predesigned model, to form a 3D construct layer by layer. Another biofabrication method may further include seeding the porous construct with the aforementioned cells after printing. In another embodiment, the cells may be mixed with the biomaterial prior to printing such that the cells are incorporated directly into a construct during the printing process (which occurs prior to crosslinking).

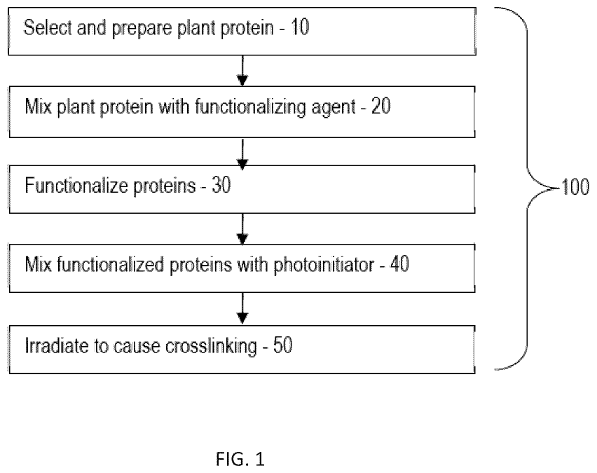

FIG. 1 is a flow chart that depicts an exemplary embodiment of a method for fabricating biomaterials 100 as disclosed herein. In a first step 10, plant materials are selected. Preparation is undertaken and may include various forms of reduction including grinding, defatting, percipitation, protein extraction, and as otherwise described herein. In a second step 20, the reduced plant proteins are mixed with a functionalizing agent. In a third step 30, the proteins are functionalized. As set forth elsewhere herein, this may include the use of a buffered solution to enhance control of pH. This step may also include dialysis or other purification routines. In a fourth step 40, the functionalized proteins are mixed with a suitable photoinitiator. In a fifth step 50, the mixture is irradiated (that is, illuminated with photonic energy adequate to cause a desired degree of crosslinking in the mixture).

### Composition

The composition can include biomaterials, an optional photoinitiator package, an optional solvent, or a combination thereof. It is to be noted that all weight percents and weight percents per unit volume detailed below exclude the weight or volume of solvent and only take into account the weight of the solid content of the reaction product that is used to manufacture a construct. Volume to volume (v/v) ratios are those where the numerator is the volume of the primary liquid and the denominator is the total volume (the primary liquid volume and the secondary solvent volume, which is usually a buffer or water).

It is desirable for the biomaterial(s) used in the construct to have a controllable mechanical strength to modulate the cellular activity on and inside the construct, based on the mechanical properties, and also to withstand further processing. For example, in regenerative applications, the engineered construct should be handleable and implantable in the body of the living being. On the other hand, the biomaterial should be capable of imparting a texture to a food product when it is used in food production applications. It is also desirable for the biomaterials to be compatible (i.e., non-toxic) with the cells. For example, when the biomaterial is used for tissue engineering with cells interfaced or encapsulated in the biomaterial, it is desirable for it to be non-toxic. As an implant, the biomaterial has to be compatible with the body of the living being into which they are installed. The biomaterials can include plant proteins, plant fats, plant carbohydrates, plant fibers, or the like, or a combination thereof. The biomaterials preferably include plant proteins that preferably include abundant amino and hydroxyl groups.

Plant proteins can be extracted from legumes (e.g., lentils, chickpeas (garbanzo beans), black beans, kidney beans, soy products, or the like, or a combination thereof), soy products (e.g., tofu, tempeh, edamame, soy milk, or the like, or a combination thereof), nuts and seeds (e.g., almonds, peanuts, sunflower seeds, pumpkin seeds, hemp seeds, chia seeds, flax seeds, or the like, or a combination thereof), whole grains (e.g., quinoa, brown rice, barley, oats, or the like, or a combination thereof), green vegetables (e.g., spinach, broccoli, brussels sprouts, peas, or the like, or a combination thereof), sprouts (e.g., alfalfa sprouts, mung bean sprouts, or the like, or a combination thereof), or a combination thereof.

The biomaterial proteins include globulin, glutelin, legumin, vicilin, gliadin, glutenin, zein, cupin, ovalbumin, conalbumin, legumelin, avenin, 1S albumin, 2S albumin, crambin, kafirin, or the like, or a combination thereof. A preferred plant protein is 1S albumin, 2S albumin, or a combination thereof.

In an embodiment, the composition prior to crosslinking may include two or more plant proteins that are chemically different from each other. These proteins may form an interpenetrating network upon undergoing crosslinking. The resulting crosslinked material may therefore include two or more independent networks that penetrate into one another (interpenetrate) without being substantially covalently or ionically bonded to each other. In an embodiment, the resulting crosslinked material may therefore include three or more independent networks that interpenetrate into one another without being substantially covalently or ionically bonded to each other.

In an embodiment, it is desirable for the biomaterial to include the plant protein in an amount of greater than 40 wt %, preferably greater than 50 wt %, preferably greater than 60 wt %, preferably greater than 70 wt %, and more preferably greater than 80 wt %, based on a total weight of the biomaterial used in the composition.

The biomaterials are then functionalized with a functionalizing agent that includes reactive groups. The reactive groups can facilitate free-radical polymerization when activated by electromagnetic radiation, heat transfer, or a combination of electromagnetic radiation and heat transfer. The functionalizing agent preferably includes an unsaturated carboxylic acid or a derivative of an unsaturated carboxylic acid. Examples of unsaturated carboxylic acids include maleic acid, fumaric acid, itaconic acid, acrylic acid, methacrylic acid, crotonic acid, malonic acid, succinic acid, glutaric acid, adipic acid, pimelic acid, suberic acid, azelaic acid, sebacic acids, citraconic acid, or the like, or combinations thereof. Examples of derivatives of unsaturated carboxylic acids are maleic anhydride, acrylic anhydride, methacrylic anhydride, citraconic anhydride, itaconic anhydride, malonic anhydride, succinic anhydride, glutaric anhydride, adipic anhydride, pimelic anhydride, suberic anhydride, azelaic anhydride, methyl acrylate, methyl methacrylate, ethyl acrylate, ethyl methacrylate, butyl acrylate, butyl methacrylate, glycidyl acrylate, glycidyl methacrylate, or the like, or a combination thereof. A preferred reactive group for functionalizing the biomaterial is an acrylic anhydride, a methacrylic anhydride, or a combination thereof.

In a preferred embodiment, the synthesis of the functionalized biomaterial is based on the methacrylation of amino and hydroxyl groups in the protein structure. Due to the high level of amino acids in different plant legumes, nuts, grains, and seeds, various amino and hydroxyl groups can be found in their structures, making them suitable candidates for functionalization with methacryloyl groups.

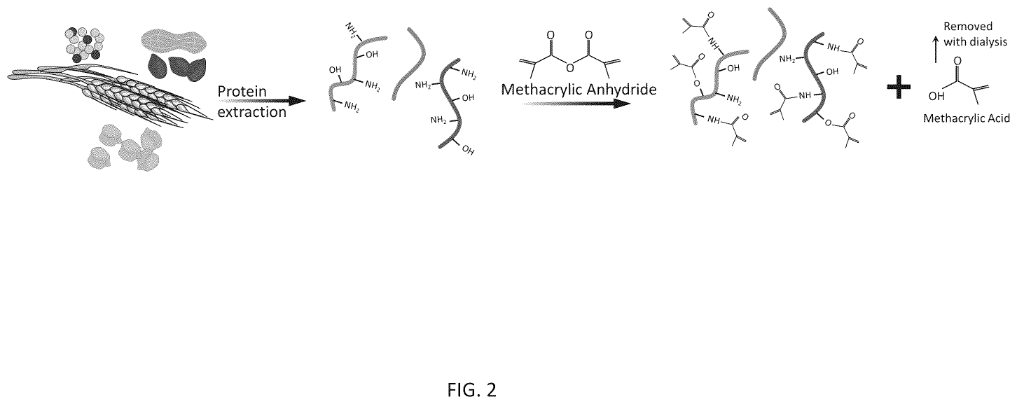

FIG. 2 is a depiction of an exemplary production process for the manufacture of photo-crosslinkable plant-derived biomaterials. Synthesis of the photo-crosslinkable plant-derived biomaterials (also referred to herein as “biomaterials”) is based on the methacrylation of amino and hydroxyl groups in protein structures. Due to the high level of amino acids in different plant legumes, nuts, grains, and seeds, various amino and hydroxyl groups can be found in their structures, making them ideal candidates for functionalization with the methacryloyl group. In some embodiments, methacrylic anhydride was used to synthesize photo-crosslinkable plant-derived biomaterials.

The functionalization reaction can be accompanied by using a pH modulation package (also referred to herein as a buffering agent). Due to the sensitivity of plant proteins to pH, controlling pH in a suitable range is desirable. The solubility of plant proteins in aqueous media increases with pH. However, a pH greater than 10 can damage the proteins. On the other hand, in a pH lower than 7, the proteins may start to precipitate, while a pH close to or lower than 4 may result in prompt precipitation and therefore limited functionalization. Furthermore, the acidic byproducts of the functionalization reaction (e.g., methacrylic acid), reduce the pH of the environment dramatically over time, therefore, continuous monitoring and adjustment of the pH is desirable for a high-quality product.

To resolve this issue, a one-pot photo-crosslinkable plant-based biomaterial synthesis process is developed and includes a buffering agent for controlling pH. A carbonate/bicarbonate buffer, with optimized molarity and target pH, is used as the solution in the synthesis protocol. The ratio of carbonate to bicarbonate was adjusted to keep the pH above 7 during the synthesis (7.5 to 8.5 was targeted), while the molarity of the buffer was adjusted based on the concentration of reactive groups (since this determines the amount of acid released in the crosslinking reaction).

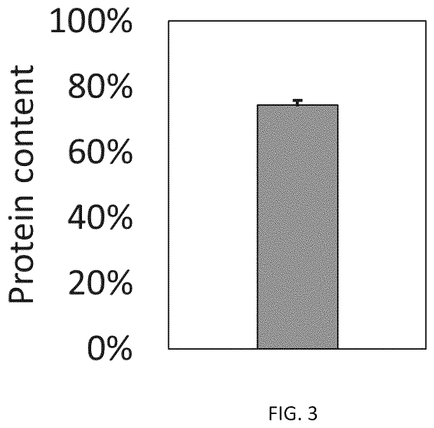

As shown in FIG. 2, proteins were first extracted from ground plant materials (legumes, nuts, grains, seeds, leaves, stems, roots, flowers, fruits, or the like, or a combination thereof). Extraction was performed in a temperature (about 40° C.) and pH (˜8.5) controlled environment. Extracted proteins were then functionalized through reaction with methacrylic anhydride in a temperature (about 0 to about 4° C.) and pH (˜7.5-8.5) controlled environment. After functionalization, the materials were dialyzed for three (3) days to remove unreacted methacrylic anhydride, salts in the buffers, and methacrylic acid. It has been demonstrated that following methacrylation and dialysis the protein content of the material remains greater than 70 wt %, while the rest of the contents include remaining fibers, carbohydrates, and fats from the protein extraction process (as depicted in the graph of FIG. 3). Sodium dodecyl-sulfate polyacrylamide gel electrophoresis (SDS-PAGE) protein staining demonstrates the presence of different bands, corresponding to different proteins including albumins, globulins, and glutelins in a chickpea-derived biomaterial before (i) and after (ii) functionalization. FIG. 3 is a bar graph that depicts protein content in the biomaterial after functionalization.

In an embodiment, a higher level of functionalization results in a lower minimum concentration of biomaterial that may be used for crosslinking (formation of a solid construct upon light exposure). In other words, the amount of crosslinkable protein used in the biomaterial may be reduced depending upon the level of functionalization. The amount of crosslinkable protein present in the biomaterial is inversely proportional to the level of functionalization.

This also implies that with a constant biomaterial concentration, certain mechanical properties (e.g., stiffness, elastic modulus) of the biomaterial with higher levels of functionalization are higher than the one with lower levels of functionalization. Furthermore, the stiffness of the construct may be varied by changing the concentration of the functionalized biomaterial in the composition. The functionalized protein is present in the composition in an amount of 10 wt % to 100 wt %, preferably 20 to 80 wt %, based on the total weight of protein in the composition.

The biomaterial may be functionalized with a functionalizing agent in an amount of 0.1 to 20 v/v % (volume of functionalizing agent solution to volume of the protein extract expressed as a percentage), preferably 0.5 to 10 v/v %, and more preferably 1 to 4 v/vt %, in order to functionalize all reactive moieties on the protein present in the biomaterial.

In an embodiment, the composition may include an optional photoinitiator package. The photoinitiator package is generally used when the biomaterials are cured (crosslinked) using ultraviolet, visible light or infrared radiation. If the biomaterials are crosslinked with other forms of radiation (e.g., xrays, electron beam, microwave radiation, radiofrequency radiation, or a combination thereof) then a photoinitiator package may be optional. If radiation is not used to effect the crosslinking, the composition may include an initiator package that is activated by thermal energy (i.e., through heat transfer). These initiators can be thermal, redox, ionizing, electrochemical, plasma, sonication, enzymatic initiators, ternary initiators, or a combination thereof, to induce crosslinking upon free radical generation upon being activated by stimuli other than electromagnetic radiation.

The photoinitiator package preferably includes a photoinitiator that can be activated in the ultraviolet to visible blue light regime of the electromagnetic spectrum. Examples of photoinitiators that work in the ultraviolet regime include benzoin methyl ether (BME), benzoin ethyl Ether (BEE), camphorquinone (CQ), DAROCUR™ series (e.g., DAROCUR™ 1173 and DAROCUR™ 2959, are benzoin methyl ether IRGACURE™ series (IRGACURE™ 184 and IRGACURE™ 907, are benzoin ethyl ether derivatives), lapachol, benzoin isobutyl ether (BIE), benzoin butyl ether (BBE), 2,2-dimethoxy-2-phenylacetophenone (DMPA), benzoin O-methyl ether (BOM), thioxanthone, anthraquinone, phenylbis(2,4,6-trimethylbenzoyl)phosphine oxide (BAPO), 2-methyl-4′-(methylthio)-2-morpholinopropiophenone (DMMMP), lithium phenyl-2,4,6-trimethylbenzoylphosphinate (LAP), or the like, or a combination thereof.

Preferred photoinitiators include camphorquinone, with peak activity around 470 nm; lithium phenyl-2,4,6-trimethylbenzoylphosphinate (LAP), with peak activity around 370 nm, and IRGACURE™ 2959 with peak activity at 280 nm. The concentration of the photoinitiators used is generally much lower than a concentration that is toxic to personnel who handle the biomaterial, to encapsulated cells or to the host recipient of a biofabricated graft. In other words, the concentration is used in biocompatible quantities—in an amount effective to facilitate the development of the appropriate mechanical and thermal properties in the construct, while at the same time not enabling any damage to personnel who handle the biomaterial, encapsulated cells or to the host recipient of a biofabricated graft. For example, LAP is used at a concentration of 0.06 to 0.3% (weight per unit volume (w/v)), while up to 0.5% w/v is recognized as being biocompatible by the United States Food and Drug Administration (FDA).

The photoinitiator is generally present in the lowest concentration that is effective to yield effective mechanical properties in the construct. This will be dependent upon the desired use for the construct. In an embodiment, any of the foregoing photoinitiators may be used in amounts of 0.01 to 0.5% weight per unit volume (w/v), preferably 0.05 to 0.4 w/v, and more preferably 0.1 to 0.3 w/v of the composition.

In another embodiment, the composition may be solubilized or suspended in an optional solvent to form a bioink that can be ejected from a nozzle in a manufacturing process. When suspended, the biomaterial is in the form of a colloidal suspension where the biomaterial particles are of a size in the nanometer range. The colloidal particles contact each other upon being ejected from the nozzle to form the construct. In an embodiment, the bioink is prepared with a suitable non-toxic, environmentally friendly solvent that can be injected from a nozzle to form the construct via an additive manufacturing process.

Volatile organic solvents may be used so long as the construct is substantially devoid of the solvent prior to its being used in the body of a living being. The evaporation of the solvent may be accomplished by drying the construct under a vacuum and/or at an elevated temperature (at a temperature greater than the boiling point of the solvent but below the flow temperature of the polymer). In another embodiment, the solvents may be washed out of the construct. Suitable solvents for producing the ink are alcohols (e.g., methanol, ethanol, propanol, butanol, or the like, or a combination thereof), water, propylene carbonate, ethylene carbonate, butyrolactone, acetonitrile, benzonitrile, nitromethane, nitrobenzene, sulfolane, dimethylformamide, N-methylpyrrolidone, acetonitrile, nitromethane, benzene, toluene, methylene chloride, carbon tetrachloride, hexane, diethyl ether, tetrahydrofuran, or the like, or a combination thereof. Water and ethanol are preferred solvents.

In an embodiment, the solvent is an aqueous solution. A preferred aqueous solution is saline. Saline refers to a solution of salt (sodium chloride) in water. It is an electrolyte solution that closely matches the electrolyte composition of human body fluids, such as blood and tears.

In an embodiment, the biomaterial is used in the bioink in an amount of 5 to 40 wt %, preferably 6 to 20 wt %, and more preferably 7.5 to 15 wt %, based on a total weight of the ink prior to being extruded through the nozzle during the additive manufacturing process to prepare the construct.

Other additives may be added to the composition depending upon the purpose that the construct is used for. In an embodiment, the other additives include a blowing agents, fillers (e.g., nanoparticles (0.5 to 100 nanometers) or microparticles (100.1 nanometers to 10 micrometers) of metal, metal oxides, bioglasses, radiopaque agents, antibacterial compounds and agents, antibiotics, bioceramics, ceramics, oxygen generating materials, proteins, vitamins, lipids, phospholipids, fatty acids, biological factors, polysaccharides, nucleic acids, growth factors, liposomes hydroxyapatite, carbon nanotubes, quaternary ammonium compounds, graphene, graphene oxide, carbon derived materials, liquid crystals, peptides, chitosan, silver nitride, platelet rich plasma, blood derived materials, food additives, heme protein, hemoglobin, myoglobin, taste and smell molecules, dyes, other plant-derived materials), antioxidants, antiozonants, mold release agents, thermal stabilizers, glass beads, elastomers, impact modifiers, gelling agents, or a combination thereof.

Blowing agents may optionally be used to convert the composition to a foam or to modify the cellular properties of a foam. Blowing agents include physical blowing agents and/or chemical blowing agents. Examples of suitable physical blowing agents may be methyl fluoride, methyl chloride, difluoromethane, methylene chloride, perfluoromethane, or the like; hydrocarbons such as acetylene, ammonia, butane, butene, isobutane, isobutylene, propane, dimethylpropane, ethane, methane, trimethylamine, pentane, cyclopentane, hexane, propane, propylene, alcohols, ethers, ketones; or the like, or a combination thereof. Chemical blowing agents include azodicarbonamide, peroxides in liquid and solid form, and other nitrogen-based organic polymers for the biomaterials.

The blowing agents are optional and may be used in amounts of 0.5 to 10 wt %, based on a total weight of the composition if desired.

In an embodiment, other synthetic organic polymers may be added to the composition. Examples of synthetic organic polymers include a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or a combination thereof. The organic polymer can also be an oligomer, a homopolymer, a copolymer, a block copolymer, an alternating block copolymer, a random polymer, a random copolymer, a random block copolymer, a graft copolymer, a star block copolymer, a gradient polymer, a dendrimer, a polyelectrolyte (polymers that have some repeat groups that contain electrolytes), a polyampholyte (a polyelectrolyte having both cationic and anionic repeat groups), an ionomer, or the like, or a combination comprising at last one of the foregoing organic polymers. The organic polymers have number average molecular weights greater than 10,000 grams per mole, preferably greater than 20,000 g/mole and more preferably greater than 50,000 g/mole.

Examples of thermoplastic synthetic polymers include a polyacrylic, a polycarbonate, a polyalkyd, a polystyrene, a polyolefin, a polyester, a polyamide, a polyaramid, a polyamideimide, a polyarylate, a polyurethane, an epoxy, a phenolic, a polysiloxane, a polyarylsulfone, a polyethersulfone, a polyphenylene sulfide, a polysulfone, a polyimide, a polyetherimide, a polytetrafluoroethylene, a polyetherketone, a polyether ether ketone, a polyether ketone ketone, a polybenzoxazole, a polyoxadiazole, a polybenzothiazole, a polybenzothiazinophenothiazine, a polypyrazinoquinoxaline, a polypyromellitimide, a polyguinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a polysulfonamide, a polyurea, a polyphosphazene, a polysilazane, a polyolefin, or the like, or a combination thereof.

Examples of thermosetting polymers suitable include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination thereof.

In an embodiment, the composition may contain biodegradable polymers. Suitable examples of biodegradable polymers are as polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or the like, or a combination thereof.

The synthetic organic polymers and/or the biodegradable polymers are optional and may be used in amounts of 0.5 to 10 wt %, based on a total weight of the composition if desired.

In an embodiment, gelling agents may be used in the composition as well. Gelling agents may provide elastic reinforcement to the crosslinked reaction product. Examples of gelling agents include gelatins, alginates, carrageenans, oligosaccharides, polysaccharides, or a combination thereof. Examples of gelling agents added to the composition are hydrocolloids, examples of which are vegetable gums, a pectin blend that includes one or more of konjac, xanthan, pectin, locust bean gum and/or agar. Examples of other gelling agents that may be used include gelatin, gellan gum, carrageenan, guar gum, psyllium seed gum, yam starch powder, alginate, seaweed flour, tragacanth gum, karaya gum, curdlan, soy polysaccharides, alginic acid, carboxymethylcellulose, agar-agar, carrageenans, locust bean gum, gelatin, alginate, arabinoxylan, arrowroot, cassia gum, cellulose, gum Arabic, karaya gum, konjac, kuzu, maltodextrin, marshmallow root, pectin, sodium alginate, starch, xanthan gum, b-glucan or a combination thereof.

The gelling agents are optional and may be used in amounts of 0.5 to 10 wt %, based on a total weight of the composition if desired.

### Manufacturing of the Composition

It is desirable to have the composition contain a plant-based protein in a target concentration that is desirable for a particular application. For example, it is desirable to have a minimum concentration of crosslinks for cells that are encapsulated in a 3D construct, while maintaining a higher concentration of crosslinks for cells seeded on the construct. On the other hand, a higher crosslink concentration is desirable for handleability or for texture when the construct is used for food production.

The plant-based protein is preferably one that has a high concentration of amino and hydroxyl functionalities. The amino acid profile of the plant-based protein should be good for cell activity and food nutritional value. The biomaterials (including plant proteins, with residual plant fats, plant carbohydrates, and plant fibers) are first subjected to a functionalization reaction with the functionalizing agent to produce a functionalized biomaterial. Upon functionalization, unreacted chemicals and byproducts are removed from the functionalized biomaterial through a separation process. This is a purification process and does not result in the removal of plant proteins, plant fats, plant carbohydrates, and plant fibers from the functionalized biomaterial. The separation process may include dialysis, centrifugation, filtration, chromatographic separation, or the like, or a combination thereof to provide a biomaterial with a protein content that is greater than 40 wt %, greater than 50 wt %, greater than 60 wt %, preferably greater than 70 wt % and more preferably greater than 80 wt %, based on a total weight of the biomaterial. The remainder of the biomaterial can be plant fats, plant carbohydrates, and plant fiber.

An optional photoinitiator package, a pH modulation package, and an optional solvent can then be added to the biomaterials to produce the composition. The biomaterials with the high protein content can be reacted to form a crosslinked network upon exposure to light in the presence of a photoinitiator with a peak light absorption close to the wavelength of light.

The photoinitiator package is optional depending upon the type of radiation used to crosslink the biomaterial. For example, the photoinitiator package may not be used in the composition if forms of radiation other than ultraviolet or visible light radiation are used to facilitate crosslinking. The solvent may also be optional depending upon the viscosity of the composition. If the viscosity is low, then there may not be a need to use the solvent to manufacture a bioink.

The functionalized biomaterial, the photoinitiator package, the pH modulation package, the optional solvent are mixed in a mixer to form the composition. The composition with the solvent is also called a bioink and may be used in an additive manufacturing process to produce articles. These will be described later.

In an embodiment, the mixing may be conducted by vortex mixing in a Waring blender, a Henschel mixer, a static mixer, an extruder, or the like. The composition may then be poured into a mold and subjected to electromagnetic radiation to form the construct. The composition may be loaded into a syringe and printed using an extrusion-based printing. The composition may be loaded into a bath and crosslinked selectively using a steriolithography-based printing. The composition may be injected in vivo without a regular shape and crosslinked in situ.

In an embodiment, the mixing may be conducted at a rotational velocity effective to form an emulsion using another liquid phase, a solid phase, a gas phase, or a combination thereof. The rotational velocity of the mixer may be 100 to 15000 revolutions per minute. The rotational velocity is high enough to beat ambient air or an inert gas into the composition and to form a liquid foam. The liquid foam is poured into a mold in this foamed state and crosslinked using radiation and/or thermal energy to produce a solid foam, which is used as a construct. The gas can be mixed with the solution using other methods such as air bubble injection or cavitation.

In another embodiment, the liquid (prior to foaming) or the liquid foam may be transferred from the mixer to an additive manufacturing device, where it is ejected from a nozzle as a bioink and cured to form the solid foam, which is used as the construct.

### Manufacturing of the Construct

The composition detailed above may be subjected to fabrication to produce a construct into which cells can be disposed to form a prosthetic. The composition may be used as a liquid without foaming or with foaming to form a solid crosslinked construct. The liquid (in unfoamed or foamed form) may be subjected to further processing using extrusion, 3D bioprinting, stereolithography, molding, surface patterning, spinning, electrospinning, wet spinning, self assembly, emulsification, or a combination thereof.

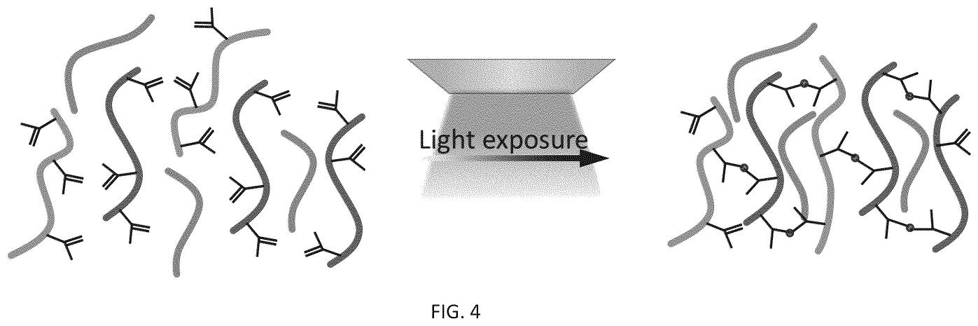

In an exemplary embodiment, the construct is used as a scaffold to incubate a variety of cells that may eventually be used to replace damaged tissue or tissue that has been removed. It may also be used for the development of food products. In an embodiment, the composition may be emulsified during mixing and the resulting foam poured into a mold having the desired shape. The mold may be subjected to electromagnetic radiation to facilitate crosslinking. The electromagnetic radiation may include ultraviolet radiation, visible light, microwave radiation, xray radiation, infrared radiation, electron beam radiation, radio frequency radiation, or a combination thereof. FIG. 4 depicts crosslinking of the biopolymer of FIG. 2 using electromagnetic radiation.

When ultraviolet or visible light is used, it is desirable for the composition to contain the photoinitiator package. The photoinitiators undergo a chemical reaction upon exposure to ultraviolet radiation or visible light, and they generate a reactive species (such as free radicals or cations), leading to the formation of crosslinks between polymer chains. Ultraviolet radiation is typically in the range of 100 to 400 nanometers. Visible light is typically in the range of 380 to 700 nanometers. The generated reactive species initiate the crosslinking reaction by forming covalent bonds between adjacent polymer chains. As crosslinks are formed, the material undergoes curing and hardening. This transformation can occur rapidly, often in a matter of seconds or minutes, depending on the ultraviolet radiation intensity and the specific formulation of the composition.

When microwave radiation, radiofrequency radiation, and infrared radiation are used, an initiator package that is devoid of photoinitiators may be used. Since microwave radiation, radiofrequency radiation and infrared radiation typically facilitate crosslinking by heating the composition, the initiator package may be devoid of a photoinitiator. In an embodiment, microwave radiation, radiofrequency radiation and/or infrared radiation may be used in conjunction with ultraviolet radiation to facilitate crosslinking of the composition.

When xray and/or electron beam radiation are used to crosslink the composition, an initiator package may be absent from the composition. Xray and electron beam radiation are both forms of ionizing radiation. These forms of radiation typically break molecular bonds (especially unsaturated carbon bonds present in the functionalized protein) releasing free radicals that can facilitate curing of the composition by forming covalent bonds between adjacent polymer molecules, creating a three-dimensional network. Since the free radicals are created by the breaking of molecular bonds, there is no need for the composition to contain a photoinitiator package.

Curing using ultraviolet radiation and visible light is preferred. When ultraviolet radiation, microwave radiation, radiofrequency radiation, and/or infrared radiation is used, the cells may be mixed with the liquid or a liquid foam prior to curing. When microwave radiation, radiofrequency radiation, and/or infrared radiation is used, the cells may be mixed with the liquid or liquid foam prior to curing or dripped or positioned into the solid foam after curing. When xrays or electron beam radiation are used to facilitate curing of the liquid foam to form the solid foam, the cells are added to the solid foam after it is foamed. This is done to prevent damage to the cells by the ionizing radiation.

The crosslinking of the composition results in the entrapping of cells in the foam or hydrogel to form a solid foam or hydrogel, respectively. When the liquid foam is crosslinked, it forms a crosslinked solid porous cellular construct (e.g., a solid foam). When the liquid prior to foaming is crosslinked, it typically forms a hydrogel.

The resulting crosslinked porous foam may be used as a construct. The construct preferably has open cells or a combination of open cells and closed cells. The open cells are generally larger in number than the closed cells. In the case of foam, the construct has a porosity of 20 to 80 volume percent, preferably 30 to 60 volume percent, based on a volume of the entire construct. The struts of the foam may have additional porosity (e.g., microporosity) in addition to that developed from the foaming.

The porosity of the construct may be due to both macroporosity and microporosity. Macroporosity (having an average cell size of 100 nanometers or greater) is generated through foaming. Microporosity (having an average pore size of less than 100 nanometers, preferably less than 50 nanometers) is inherent, due to the contribution of water in the liquid composition. Microporosity is the characteristic of most hydrogels, due to the contribution of the water in their structure.

The construct after crosslinking has modulus of 1 kPa to 1 MPa measured under compression or measured in tension It can be elastic or rigid depending upon the desired end application. After crosslinking, the construct can be saturated with cells that can proliferate inside the construct to form a tissue engineered construct. On the other hand, the cells can be encapsulated in the liquid form and immobilized upon crosslinking in a 3D construct. These cells are able to spread, proliferate, and migrate inside and outside the crosslinked construct over time. The various types of cells are listed above and will not be elaborated upon here in the interests of brevity. In an embodiment, the cells can be therapeutic autologous cells. The construct with the therapeutic cells may then be disposed of in the body of a living being to facilitate the regeneration of damaged tissue or to replace tissue that has been removed.

In an embodiment, the liquid may be used as a bioink and used in an additive manufacturing process to form the construct. The therapeutic cells may be added to the composition in liquid form or in liquid foam form to form the bioink, which is then used in additive manufacturing. As noted above, in an additive manufacturing process objects are built layer by layer, based on a digital model.

In another embodiment, the additive manufacturing may include bioprinting. Bioprinting is an additive manufacturing technique that involves the precise deposition of biological materials, such as cells, biomaterials, and bioactive factors, to create functional, living tissue-like structures. In bioprinting, specialized 3D printers are used to deposit the bioink layer by layer in a controlled manner, following a digital design or blueprint (that may be provided by the computer-aided design (CAD) program). These bioinks include living cells that are suspended in the composition, providing structural support and promoting cell attachment, growth, and differentiation. Various types of cells, including stem cells, primary cells, and cell lines, can be used in the bioprinting process, depending on the desired tissue or organ being fabricated.

In an embodiment, the bioprinting may include embedding autologous cells (e.g., esophageal epithelial cells, adipose-derived mesenchymal stromal cells (Ad-MSC) in the construct.

In an embodiment, the bioprinting is conducted using ultraviolet radiation, visible light, microwave radiation, radiofrequency radiation, and/or infrared radiation. Ultraviolet radiation, infrared radiation or visible light is preferred. The bioink may be subjected to ultraviolet radiation, visible light, microwave radiation, radiofrequency radiation, and/or infrared radiation either simultaneously or sequentially with the layer-by-layer deposition of the bioink. In a preferred embodiment, the bioink may be subjected to only ultraviolet radiation either simultaneously or sequentially with the layer-by-layer deposition of the bioink.

In some embodiments, different fabrication processes are used to generate different structures. The materials may be subject to electrospinning, wet spinning or drawing to form fibrous structures. Generally, a variety of forms may be used, with most often crosslinking of the construct occurring during the formation or immediately after formation, to stabilize the structure.

The composition and the constructs manufactured therefrom display a number of advantages. The photo-crosslinkable composition disclosed herein is a product that not only can be used in research and teaching institutions but has many commercial applications in tissue engineering ranging from invitro tissue modeling to regenerative medicine and food biomanufacturing. The composition can be used for 2D or 3D culture of cells for various applications including but not limited to (i) drug discovery for various diseases including cancer, cardiac disorders, skin defects, and the like; (ii) modeling the behavior of various in vivo tissues including but not limited to heart, skin, muscle, bone, brain, and the like; (iii) recreating microbial environment for studying the behavior of different bacteria, virus, and fungi or for fermentation, recombinant product fabrication, and the like.

The biomaterial can also be used in the form of cellular/acellular construct for the regeneration of various tissues including but not limited to cardiac, skin, skeletal muscle, bone, brain, cornea, and the like. Different forms of the material (bulk, foam) mixed with other biomaterials/particles/cells can be implemented. In this case, the end user would be (i) academic researchers in research institutions (pre-doctoral researchers, post-doctoral researchers, principal investigators, etc.), mostly for in vivo studies, and (ii) procedural, wound prep, and wound management care providers in healthcare systems. The medical applications range from public hospitals, private practices, and military health care. The end user will vary from field medics, paramedics, dermatologists, plastic surgeons, wound care nurses, orthopedic surgeons, cardiothoracic surgeons, and so on.

The biomaterial may be used in the field of cellular agriculture. The composition and the constructs derived therefrom are plant-based and limit the application of animal-derived materials, which is the main goal of cellular agriculture. The protein-rich nature of the constructs reduces the need for serum supplementation, further reducing the xeno-based materials in this field. Some specific applications could be (i) in vitro 2D/3D cell culture for studying the behavior of muscle cells for food biomanufacturing, (ii) development of artificial meat with cell agriculture, and (iii) development of plant-based protein alternatives in food biomanufacturing. The end users would be (i) academic or industrial researchers in this field as well as (ii) food biomanufacturing companies.

The construct can carry drugs for various applications. Commonly, 3D constructs are being used for localized and sustained delivery of bioactive agents into various tissues. The implementations would vary from cosmetic to various wound healing applications. In this case, the end users can be researchers, clinicians, and/or persons applying cosmetic biomaterials.

The chemical composition and the mechanical properties of the construct have a substantial impact on the cellular behaviors in 3D environments. In addition, the biological functional groups found in the biomaterial structure also influence cell proliferation and adherence, while pore size can influence nutrient diffusion, cell migration, and growth. Furthermore, the 3D construct can be tuned to degrade at a rate similar to tissue formation. The disclosed constructs meet this expectation for proper tissue integration and formation in regenerative medicine and efficient cellular growth in cellular biomanufacturing. The level of porosity in foam constructs can enhance cellular infiltration, spreading, and migration inside the tissue-engineered constructs.

The biomaterial composition prior to and after crosslinking and the method of manufacturing thereof will now be described.

### EXAMPLES

### Materials, Sample Preparation, and Methods Used

In the trials presented herein, all plant materials were produced by GOYA and purchased from Walmart. Methacrylic anhydride, lithium phenyl-2,4,6-trimethyl-benzoyl phosphinate (LAP) photoinitiator, sodium carbonate, sodium bicarbonate, hydrochloric acid, and sodium hydroxide were purchased from Sigma-Aldrich. Dulbecco's phosphate-buffered saline (DPBS, no calcium, no magnesium), Dulbecco's modified eagle medium (DMEM), fetal bovine serum (FBS), penicillin-streptomycin (PS), trypsin-ethylenediaminetetraacetic acid (EDTA), Live/Dead kit, containing green-fluorescent calcein-AM and red-fluorescent ethidium homodimer-1, and Micro BCA protein assay kit were purchased from ThermoFisher Scientific. Hexane was purchased from Fisher Scientific. C2C12 myoblasts were obtained from American Type Culture Collection.

The plant materials were reduced by being ground, defatted twice in hexane at a concentration of 25% (w/v) for 30 minutes, and dried under vacuum overnight. The defatted flour was then stored in a dry environment until use. For protein extraction, the flour was added to pre-warmed (40° C.) double-deionized water (DDW) to make a suspension with a concentration of 25% (w/v) and stirred for two hours at 40° C. and pH-8.5. The pH was adjusted using sodium hydroxide and monitored with a Mettler Toledo benchtop pH meter. The suspension was then centrifuged at 4700 grams (g) for 30 minutes (min) and the supernatant was collected as a protein isolate solution. The solution was then quenched on ice water and methacrylated by slowly and dropwise adding 0.5, 1, or 2 milliliters (ml) of methacrylic anhydride per 100 ml of the solution to synthesize a low, medium, and high level of functionalization, respectively. The pH was continuously monitored and adjusted in the range of 7.5-8.5 by adding sodium hydroxide. The methacrylation was performed for 1 hour (hr) and stopped by adding 400 milliliters (ml) of 3×DPBS to 100 ml of the solution.

The solution was then dialyzed in 3.5 or 7 KDMW cutoff tubing (Fisher Scientific) for 3 days against DDW, filtered, and lyophilized (FreeZone 2.5 L −50 C Benchtop, Labconco) for future application in the experiments. For quantification of the protein content, the lyophilized foam was dissolved in DPBS and analyzed for protein concentration using a Micro BCA protein assay kit (Thermo Fisher Scientific) with a Cytation 5 imaging reader (BioTek, Winooski, VT).

To eliminate the sophisticated continuous pH monitoring and adjustment, a one-pot strategy was developed by modifying the protein extraction and functionalization described before. For this purpose, a carbonate/bicarbonate buffer was replaced with DDW in the previous protocol. Sodium bicarbonate and sodium carbonate were mixed at a mass ratio of 8:1 and dissolved in DDW at a molarity of 0.125, 0.25, and 0.5 for low, medium, and high functionalization, respectively. The buffer was then used to prepare plant flour suspension for protein extraction.

After protein extraction, the supernatant which contains the carbonate/bicarbonate buffer used in the functionalization step. The solution was dialyzed, filtered, lyophilized, and stored in a dry environment for experiments.

To evaluate the minimum crosslinkable weight to volume concentration, a tube inversion test was implemented. The lyophilized photo-crosslinkable plant-derived material was dissolved in DPBS at different concentrations and LAP was added at a concentration of 0.3% weight per unit volume (w/v) to the solution to prepare the precursor. 0.5 ml of the precursor was transferred to a glass vial and exposed to blue light (405 nm) for 1 min. The tubes were then inverted to investigate if the material is crosslinked (solidified).

For mechanical properties, Young's moduli were measured using a Mach-1 micromechanical tester (Biomomentum) equipped with a 2 millimeter (mm) spherical probe. The precursor was transferred to a polydimethylsiloxane (PDMS) cylindrical well (10 mm diameter, 4.5 mm height) and photo-crosslinked for 1 min. The test was then performed by submerging the sample and probe in DDW and the results (n=9) were presented as mean±standard deviation.

For scanning electron microscopy (SEM) analysis, biomaterial precursors with different concentrations were prepared in DPBS (as described before), transferred to a PDMS cylindrical well (10 mm diameter, 4.5 mm height), and photo-crosslinked for 1 min. The sample was then dropped into a liquid nitrogen bath to snap-freeze the hydrogel and was immediately placed inside the freeze dryer to lyophilize for 24 hours (h). The sample was then broken to expose the cross-section for imaging the internal structure of the construct. The cross-section was mounted on a stub and coated with gold using a sputter coater device (Vacuum Desk V, Denton) for 60 s at 20 mA. A benchtop SEM (TM-1000, HITACHI) was then used to capture the images.

The cells were sub-cultured in Dulbecco's Modified Eagle Medium supplemented with 10% volume per unit volume (v/v) fetal bovine serum (FBS) and 1% (v/v) penicillin-streptomycin (PS) at 37° C. in a humidified atmosphere containing 5% CO₂. Cells at passage number 8 were used in the experiment. To assess the biocompatibility, a biomaterial precursor was prepared as described before, transferred to a 48-well plate, and photo-crosslinked in the wells. The cells were then detached and resuspended in growth media, followed by their seeding on the construct. A live/dead assay was performed one day after seeding by incubating cells for 15 min at 37° C. with a solution of 2 microliters per milliliter (μl/ml) ethidium homodimer-1 and 0.5 μl/ml calcein AM in DPBS. Fluorescence Microscopy was performed on a Zeiss Observer D1 microscope. Quantification of the viability data was done using 4 samples and the results were presented as mean±standard deviation.

Various other components may be included and called upon for providing for aspects of the teachings herein. For example, additional materials, combinations of materials, and/or omission of materials may be used to provide for added embodiments that are within the scope of the teachings herein.

### Example 1

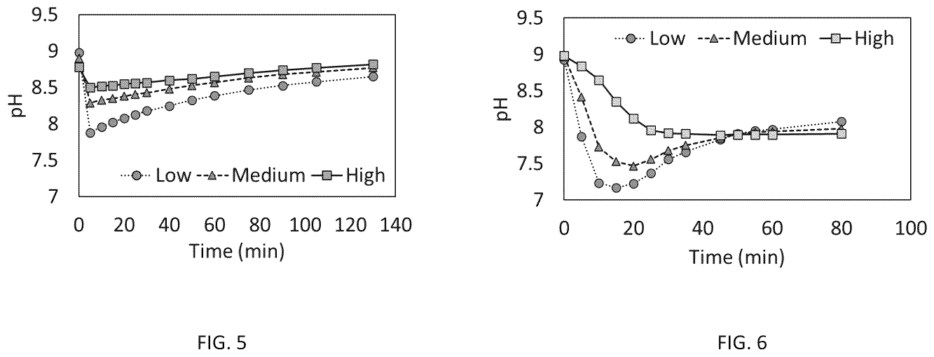

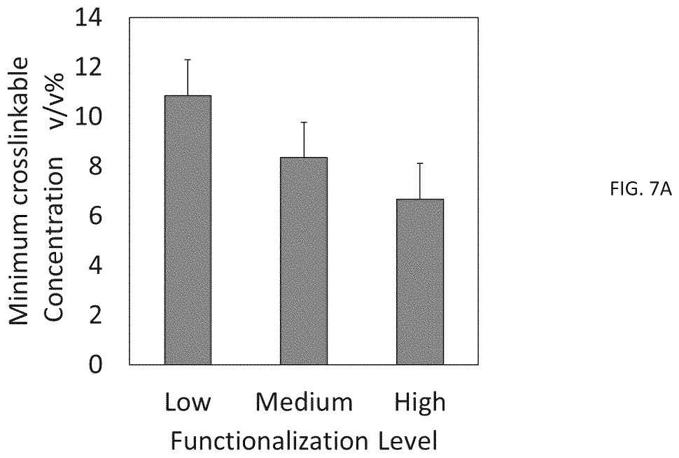

This example was conducted to demonstrate the effect of a carbonate/bicarbonate buffer, (with optimized molarity and target pH) on the solution in the synthesis protocol. The ratio of carbonate to bicarbonate was adjusted to keep the pH above 7 during the synthesis (pH of 7.5 to 8.5 was targeted), while the molarity of the buffer was adjusted based on the concentration of methacrylic anhydride. FIGS. 5 and 6 demonstrate the capability of carbonate/bicarbonate buffer at different molarities to adjust the pH to be in a desirable range during the extraction (FIG. 5) and functionalization (FIG. 6) process. Through photo-crosslinking of different concentrations of biomaterials derived from functionalization with or without buffer solution, it is confirmed that there was no significant difference between the materials synthesized with manual pH adjustment and automatic pH buffering using the buffer solution.

That is, with regard to FIGS. 5 and 6, a carbonate/bicarbonate buffer with optimized molarity and target pH was used for maintaining the pH above 7. FIG. 5 shows pH change over time during the protein extraction process, while FIG. 6 shows pH change over time during the protein functionalization process. For both of the steps, the pH was maintained at a value greater than 7, and materials derived from the synthesis did not show a significant difference between the materials derived from functionalization with manual pH adjustment. Low, medium, and high demonstrate the molarity of the buffer (0.125, 0.25, and 0.5) and the corresponding concentration of methacrylic anhydride (0.5, 1, and 2 (% w/v)) in the functionalization step.

### Example 2

This example was conducted to demonstrate that the mechanical properties and microstructure of the plant-based constructs could be tailored (tuned) to a desirable value. Here, methacrylic anhydride in amounts of 0.5%, 1%, and 2% (v/v) concentration was used to functionalize the extracted protein solution, respectively for a low, medium, and high degree of functionalization. Chickpea was used as an exemplary plant material for these experiments. As further proof, the peaks corresponding to lysine protons (around 3 ppm), corresponding to non-functionalized groups, were showing a decreasing rate by increasing the level of functionalization, with a high level of functionalization not showing any peak, demonstrating a full level of functionalization.

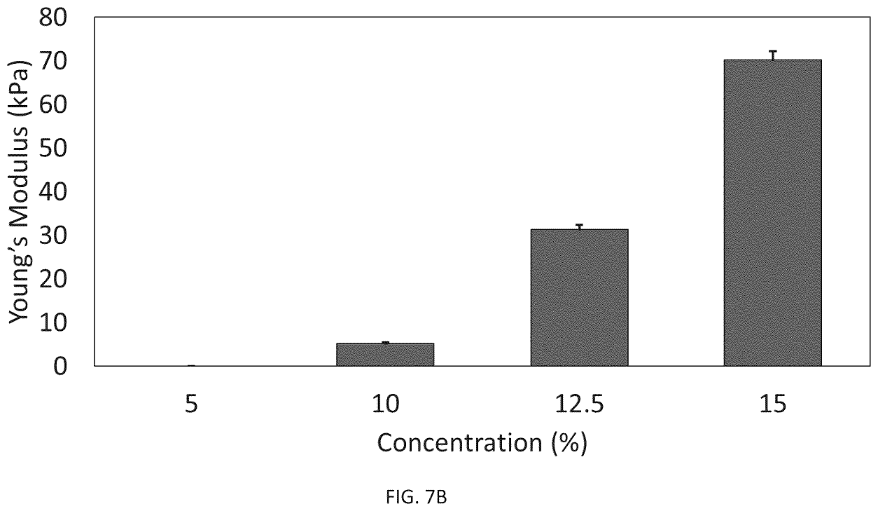

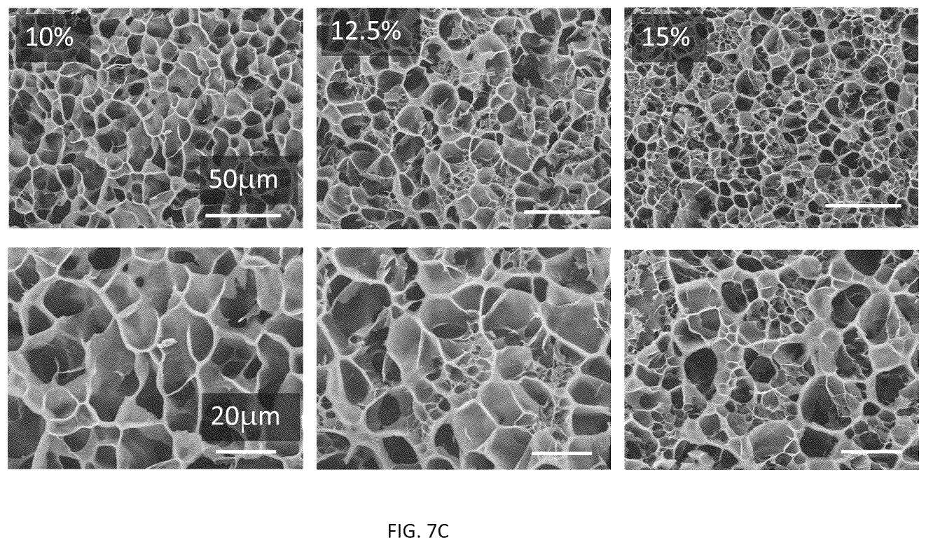

Here it is demonstrated that the synthesized biomaterials are not only crosslinkable but also modulatable to form constructs with tailored mechanical properties for various tissue engineering applications (See FIG. 7A). The results demonstrated that the level of functionalization could be adjusted to modulate the crosslinking. As shown in the bar graph of FIG. 7A, a higher level of methacrylation results in a lower concentration of biomaterial that may be used for crosslinking (formation of a solid construct upon light exposure). This means that with a constant concentration of the biomaterial in the composition, the mechanical properties of the biomaterial with higher levels of functionalization are higher than the ones with lower methacrylation.

These findings were supported by results obtained by proton nuclear magnetic resonance (1H NMR) analysis (not shown here). The peaks corresponding to methacrylamide protons (around 5.75 ppm)(not shown here), which correspond to the functionalized groups were enlarged with increasing the level of functionalization. However, by increasing the concentration of methacrylic anhydride beyond 2% v/v, the functionalization level is not further increased. This means that a full functionalization of the protein at 2% v/v methacrylic anhydride could be achieved.

Furthermore, the stiffness of the construct could be adjusted by changing the concentration of the functionalized protein in the biomaterial solution (See FIG. 7B). A Young's modulus of less than 10 kPa could be achieved with 10% w/v concentration of medium methacrylated protein to enable high cellular activity on and inside the construct. On the other hand, higher concentrations could result in stiffer constructs (around 30 kPa and 7 kPa Young's modulus for 12.5 and 15% w/v concentration of medium methacrylated protein, respectively), which can match the stiffness of different tissues in vivo. From the FIGS. 7A and 7B, it can be seen that the mechanical properties can be varied with the concentration of the functionalized protein in the composition, which makes the construct an ideal candidate for cell culture, with the potential to provide both cell permissibility (at lower concentrations) and stiffness (at higher concentrations) to match the properties of native tissues.

The analysis of the internal microstructure of the construct through scanning electron microscopy (SEM) demonstrates that the construct structure is highly porous, supporting cellular activity and enabling rapid mass transport inside the construct (FIG. 7C). Furthermore, increasing the concentration of the construct enables the formation of secondary crosslinked structures, enhancing the mechanical properties and structural stability.

### Example 3

This example was conducted to demonstrate the cellular permissibility of photo-crosslinkable plant-derived constructs (constructs derived from the compositions disclosed herein). The chemical composition and the mechanical properties have a substantial impact on the cellular behaviors in 3D environments. In addition, the biological functional groups found in the biomaterial structure also influence cell proliferation and adherence, and pore size can influence nutrient diffusion, cell migration, and growth. Furthermore, the 3D constructs must degrade at a rate similar to tissue formation. Biodegradable hydrogel-based constructs meet this expectation for proper tissue integration and formation in regenerative medicine and efficient cellular growth in cellular biomanufacturing.

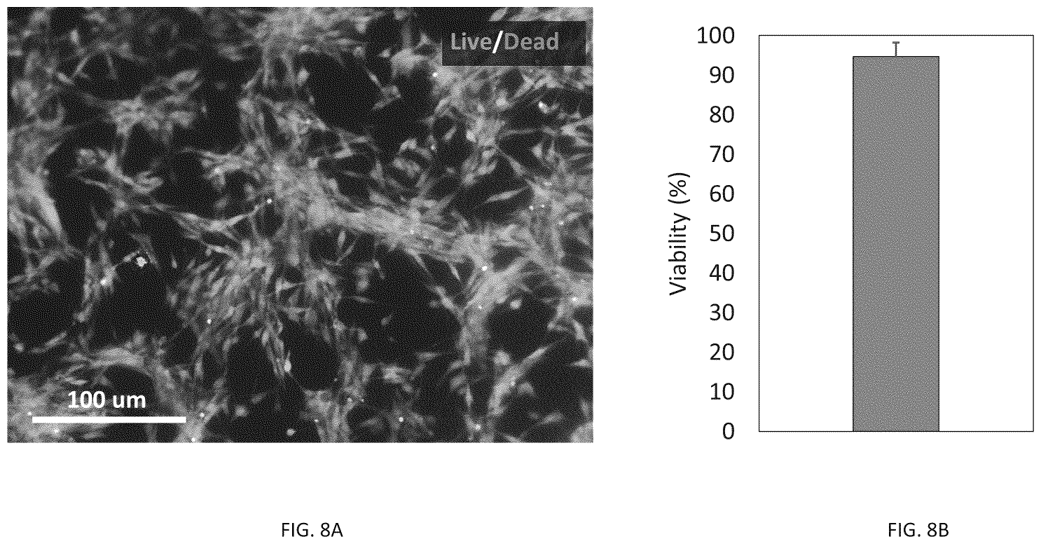

Cellular behavior was evaluated here to confirm the biocompatibility of the synthesized photo-crosslinkable plant-based constructs (FIG. 8A). The material was injected into cell culture well plates, crosslinked and myoblast cells were directly seeded on the construct without any washing. After 1 day, the cells were evaluated using live/dead staining. As shown in FIG. 8A, the cells properly attached to the material, demonstrating the presence of cell binding sites in the protein structure. Furthermore, the quantification of staining results confirmed the high level of biocompatibility (greater than 94%) of the product (FIG. 8B). FIG. 8A is a micrograph that depicts live/dead staining which demonstrates the viability and morphology of myoblasts, one day after seeding on the photo-crosslinked chickpea-derived construct. The result shows proper cellular attachment and spreading on the construct, demonstrating the biocompatibility and cell binding capability of the construct. FIG. 8B is a bar graph that depicts quantification of the stained samples thereby demonstrating a greater than 94% viability one day after post-seeding.

The normal cell morphology as well as minimal cell death demonstrates that the dialysis was performed effectively, removing the unreacted methacrylic anhydride, salts in the buffers, and methacrylic acid. The protein-rich nature of the construct further ensures the proper cell binding, spreading, and biodegradation through protease reactions.

### Example 4

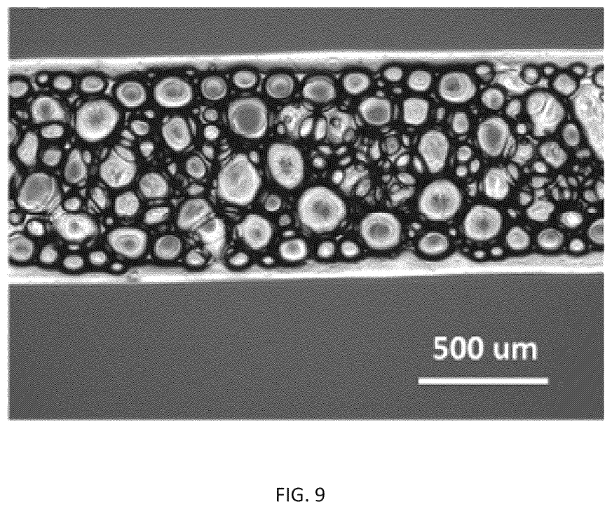

This example was conducted to demonstrate the biofabrication capabilities using photo-crosslinkable plant-derived bioinks. The cellular microenvironment plays a useful role in improving the function of micro-engineered tissues. Control of the microarchitecture in engineered tissues can be achieved through various biofabrication technologies. In this example, the capability of the material to serve as a bioink for various biofabrication processes commonly used for tissue engineering and drug delivery applications was assessed. The results demonstrated not only high level of processibility through micromolding and bioprinting, but also retaining the cellular viability during the biofabrication process (See FIG. 9). FIG. 9 is a photomicrograph that depicts the bioprinting of a photo-crosslinkable chickpea-based foam bioink.

This ability is valuable for tissue engineering, specifically when targeting the biofabrication of complex constructs. Rapid crosslinking in combination with the highly biocompatible nature of the material enables simultaneous bioprinting and in situ crosslinking, making the biomaterial an ideal candidate for in situ bioprinting. Another advantage of the developed biomaterial is that it can be used in bulk hydrogel (non-foamed) and foam form.

The foam derived from whipping plant proteins (specifically albumin) is a widely used material alternative to whipped cream for vegan foods. Here, the formation of highly porous constructs ideal for tissue engineering is demonstrated. The foam was created from bioink upon 30 sec of mixing at 15000 RPM. This stable foam was then loaded into a syringe and printed with simultaneous photo-crosslinking using ultraviolet radiation to demonstrate the biofabrication capability of the foam bioink (FIG. 9). A high level of porosity in foam constructs (as depicted in the FIG. 9) can enhance cellular infiltration, spreading, and migration inside the tissue-engineered constructs.

### Example 5

This example was conducted to demonstrate that photo-crosslinkable biomaterials (constructs) could be generated from different plant species (other than just biomaterials available from chickpeas).

While most of the results in the previous examples were generated using chickpea-derived photo-crosslinkable biomaterials, it is desirable to demonstrate that the proposed strategy is applicable for other plant species. Theoretically, as long as the plant material contains amino acids, it can be functionalized using the methods described herein (see FIG. 1) to render it photo-crosslinkable. To demonstrate that this method is not limited to chickpea (legumes), and that it can be applied to any plant material containing amino acids other widely used plant-based edible products were evaluated for functionalization. Using the same protocol described above in this disclosure, a successful functionalization of extracted proteins from other plant-based materials including nuts (peanut), grains (oat), and seeds (quinoa) were demonstrated (not shown). All of these plant-based biomaterials were crosslinked by exposure to blue light (405 nm) in the presence of a photoinitiator.

### Example 6

This example was conducted to demonstrate the culturing of cells in a 3D format (a construct). It was also conducted to characterize their behavior in the synthesized photo-crosslinkable plant-based construct.

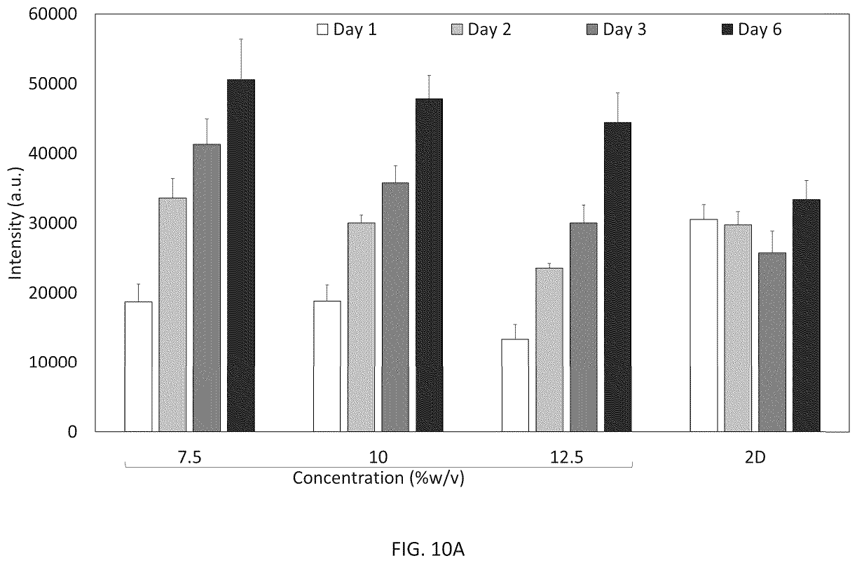

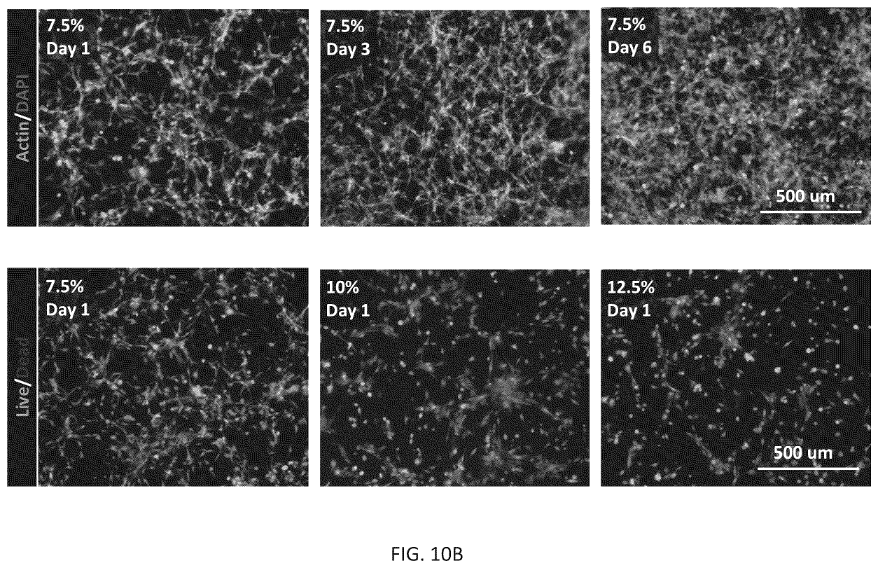

A quantitative evaluation of cellular viability and proliferation when encapsulated in 3D constructs was conducted. As seen in FIG. 10A, the cells demonstrated an increasing proliferation rate over time, while a lower concentration of the biomaterial (in the composition) better supported cellular activity. FIG. 10A is a bar graph that depicts cell proliferation in the biomaterials (3D constructs) having different protein concentrations (7.5, 10, and 12.5% w/v) in the composition as a function of time (1 day, 2 days, 3 days, and 6 days). Results are also shown for a 2D control where cells are seeded on the bottom of a well plate without a protein construct. The results show that the number of live cells in 3D culture (within the synthesized constructs) increases beyond the number of cells cultures on 2D well plate surfaces.

Qualitative representation of cellular spreading and proliferation within 3D constructs made out of photo-crosslinkable plant-derived biomaterials, over a period of 6 days, was also studied. FIG. 10B contains photomicrographs that depict live/dead staining of the cells encapsulated within the 3D construct at different concentrations of the biomaterial. Actin(green)/DAPI(blue) staining was used to investigate the cellular morphology by visualizing actin filaments and nuclei, respectively. The high level of cellular spreading and proliferation within the constructs not only demonstrates the presence of cell adhesion and biodegradation proteins in the construct but also shows the excellent biological properties of the biomaterial for tissue engineering applications, due to the high level of cellular activity and ingrowth within the constructs. The results show that all used concentrations support high levels of cellular viability.

### Example 7

This example was conducted to determine the adhesive capabilities of the biomaterial. The adhesive performance of the construct was determined in this example. The plant-derived hydrogel biomaterial (from chickpeas) is deposited on a piece of porcine tissue and attached to a methacrylated glass slide. The in-situ photo-crosslinking of the biomaterial resulted in a strong adhesion to the porcine tissue, capable of holding more than 50 g weight (equivalent to an adhesive force of 10 between the construct and 0.5 cm² (cross-sectional area) of porcine tissue).

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) an initiator for crosslinking and compositions, techniques and/or energy forms which perform that function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

What is claimed is:

1. A biomaterial composition comprising:
an uncrosslinked plant-based biomaterial functionalized with reactive groups that are operative to undergo a crosslinking reaction via free radical polymerization.

2. The biomaterial composition of claim 1, further comprising a photoinitiator package that is activated via exposure to electromagnetic radiation.

3. The biomaterial composition of claim 1, further comprising an initiator package that comprises thermal, redox, ionizing, electrochemical, plasma, sonication, ternary initiators, or a combination thereof that are operative to induce crosslinking via a free radical reaction upon being activated by stimuli that do not include electromagnetic radiation.

4. The biomaterial composition of claim 1, where the free radical polymerization induces crosslinking of the uncrosslinked plant-based biomaterial to form a crosslinked biomaterial.

5. The biomaterial composition of claim 4, where the crosslinked biomaterial is a construct that is seeded with cells after the crosslinking.

6. The biomaterial composition of claim 1, where the uncrosslinked plant-based biomaterial functionalized with reactive groups comprises at least 40% by weight of plant-based protein.

7. The biomaterial composition of claim 1, where the biomaterial composition is further mixed with cells to form a bioink prior to undergoing free radical polymerization.

8. The biomaterial composition of claim 7, where the bioink is used to biofabricate a construct; where the construct is cured either simultaneously or sequentially with the biofabrication.

9. The biomaterial composition of claim 1, where the biomaterial composition is in the form of a liquid foam prior to the free radical polymerization.

10. The biomaterial composition of claim 1, where the biomaterial composition is in the form of an unfoamed liquid prior to the free radical polymerization.

11. The biomaterial composition of claim 9, where the liquid foam is mixed with cells to form a bioink.

12. The biomaterial composition of claim 1, further comprising at least one additive, where at least one additive comprises nanoparticles, microparticles, nanofibers, microfibers, antimicrobial compounds, antibiotics, bioceramics, ceramics, oxygen-generating materials, at least one vitamin, at least one protein, at least one lipid, at least one phospholipid, at least one fatty acid, biological factors, polysaccharides, nucleic acids, growth factors, hydroxyapatite, calcium, phosphate, dopamine-based material, carbon nanotubes, graphene, graphene oxide, carbon derived materials, liquid crystals, peptides, chitosan, alginate, silver-based materials, platelet-rich plasma, blood-derived materials, food additives, heme protein, hemoglobin, myoglobin, taste and smell molecules, dyes, other plant-derived materials, antioxidants, antiozonants, thermal stabilizers, mold release agents, or a combination thereof.

13. The biomaterial composition of claim 12, wherein the additive is provided in a concentration from 0 to about 90% weight percent, based on a total weight of the construct.

14. The biomaterial composition of claim 6, where the plant-based protein comprises plant albumin, globulin, glutelin, legumin, vicilin, gliadin, glutenin, zein, cupin and/or transferrin.

15. The biomaterial composition of claim 6, where the plant-based protein is derived from legumes, nuts, grains, seeds, leaves, stems, roots, flowers, fruits, or a combination thereof.

16. A method for producing a biomaterial composition, the method comprising:
functionalizing an uncrosslinked plant-based biomaterial with reactive groups that are operative to undergo a crosslinking reaction via free radical polymerization to form a functionalized uncrosslinked plant-based biomaterial.

17. The method of claim 16, further comprising crosslinking the functionalized uncrosslinked plant-based biomaterial to form a construct; where the crosslinking occurs either prior to or after the inclusion of encapsulated cells, particles, drugs, food additives, dyes, proteins, lipids, polymers, or a combination thereof into a functionalized plant-based biomaterial.

18. The method of claim 16, further comprising foaming the functionalized, uncrosslinked plant-based biomaterial and blending it with cells to form a bioink.

19. The method of claim 18, further comprising crosslinking the bioink either sequentially or simultaneously during biofabrication.

20. The method of claim 19, where the biofabrication includes extrusion, 3D bioprinting, stereolithography, molding, surface patterning, spinning, electrospinning, wet spinning, self assembly, emulsification, or a combination thereof.
